(19) United States
(12) Patent Application Publication
(10) Pub. No.: US 2025/0011290 A1
(43) Pub. Date: Jan. 9, 2025

(54) PYRYLIUM METHOD OF PREPARING PYRIMIDINE CYCLOHEXYL GLUCOCORTICOID RECEPTOR MODULATORS

(71) Applicant: Corcept Therapeutics Incorporated, Menlo Park, CA (US)

(72) Inventors: Hazel Joan Hunt, West Sussex (GB); Gary Patrick Reid, Menlo Park, CA (US); Jeffrey Mark Dener, Menlo Park, CA (US); Aaron Dumas, Hoddesdon (GB); Stephen Hyde, Hoddesdon (GB); Conor Marrett-Munro, Hoddesdon (GB); Shaun Stairs, Hoddesdon (GB); Christopher Wise, Hoddesdon (GB)

(21) Appl. No.: 18/676,067

(22) Filed: May 28, 2024

Related U.S. Application Data
(60) Provisional application No. 63/504,823, filed on May 30, 2023.

(51) Int. Cl. C07D 239/54

(52) U.S. Cl. CPC C07D 239/54

(57) ABSTRACT

The present invention provides methods of preparing pyrimidine cyclohexyl glucocorticoid receptor modulators, methods of preparing intermediates of pyrimidine cyclohexyl glucocorticoid receptor modulators, and pyridinium compounds.

### CROSS-REFERENCES TO RELATED APPLICATIONS

This application claims the benefit of priority to U.S. Provisional Patent Application No. 63/504,823, filed 30 May 2023, the entirety of which is incorporated herein by reference.

### BACKGROUND

In most species, including man, the physiological glucocorticoid is cortisol (hydrocortisone). Glucocorticoids are secreted in response to ACTH (corticotropin), which shows both circadian rhythm variation and elevations in response to stress and food. Cortisol levels are responsive within minutes to many physical and psychological stresses, including trauma, surgery, exercise, anxiety and depression. Cortisol is a steroid and acts by binding to an intracellular, glucocorticoid receptor (GR). In man, glucocorticoid receptors are present in two forms: a ligand-binding GR-alpha of 777 amino acids; and, a GR-beta isoform which lacks the 50 carboxy terminal residues. Since these include the ligand binding domain, GR-beta is unable to bind ligand, is constitutively localized in the nucleus, and is transcriptionally inactive. The GR is also known as the GR-II.

The biologic effects of cortisol, including those caused by hypercortisolemia, can be modulated at the GR level using receptor modulators, such as agonists, partial agonists and antagonists. Several different classes of agents are able to inhibit the physiologic effects of GR-agonist binding. These antagonists include compositions which, by binding to GR, inhibit the ability of an agonist to effectively bind to and/or activate the GR. One such known GR antagonist, mifepristone, has been found to be an effective anti-glucocorticoid agent in humans (Bertagna (1984) J. Clin. Endocrinol. Metab. 59:25). Mifepristone binds to the GR with high affinity, with a dissociation constant (Kd) of 10-9 M (Cadepond (1997) Annu. Rev. Med. 48:129).

In addition to cortisol, the biological effects of other steroids can be modulated at the GR level using receptor modulators, such as agonists, partial agonists and antagonists. When administered to subjects in need thereof, steroids can provide both intended therapeutic effects, e.g., by stimulating glucocorticoid receptor transrepression, as well as negative side effects, e.g. by chronic glucocorticoid receptor transactivation. Miricorilant (CORT118335) is another such glucocorticoid receptor modulator compound, and has been described previously in PCT Publication No. WO 2012/129074, and U.S. Pat. No. 8,685,973. What is needed in the art are new methods of preparing miricorilant having lower impurity content. Surprisingly, the present invention meets these and other needs.

### BRIEF SUMMARY OF THE INVENTION

In one embodiment, the present invention provides a method of preparing a compound of Formula I:

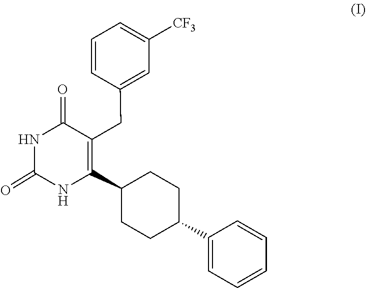 [SMILES: O=c1[nH]c(=O)c(Cc2cccc(C(F)(F)F)c2)c([C@H]2CC[C@H](c3ccccc3)CC2)[nH]1]

the method comprising:
   (a) forming a first reaction mixture comprising a first solvent, a strong acid, and a compound of Formula VII:

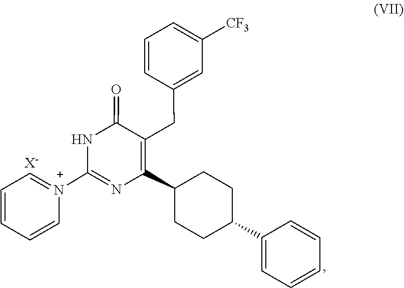

under conditions suitable to prepare the compound of Formula I,
wherein counterion X⁻ is tetrafluoroborate, chloride, bromide, tetrachloroferrate, pentachlorostannate, hexafluorophosphate, butyltriphenylborate, or tetrakis(4-methoxyphenyl)borate.

In another embodiment, the present invention provides a method of preparing a compound of Formula VII:

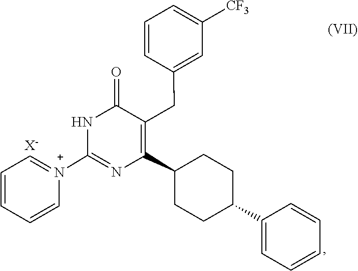

comprising
(b) forming a second reaction mixture comprising a pyrylium salt:

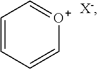

a second solvent and a compound of Formula II, or a hydrate thereof:

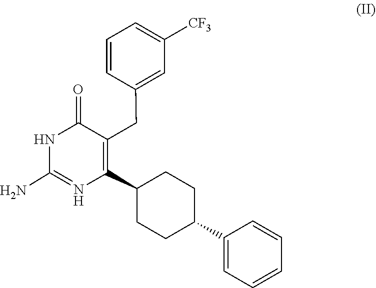 [SMILES: Nc1nc([C@H]2CC[C@H](c3ccccc3)CC2)c(Cc2cccc(C(F)(F)F)c2)c(=O)[nH]1]

under conditions suitable to prepare the compound of Formula VII,
wherein counterion X⁻ is BF₄⁻.

In another embodiment, the present invention provides a compound of Formula VII:

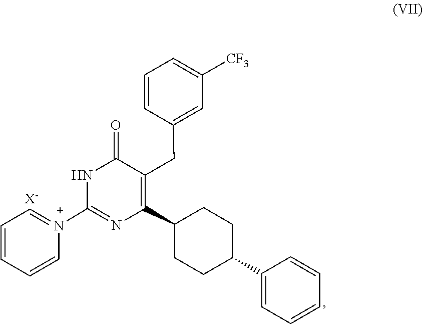

wherein counterion X⁻ is tetrafluoroborate, chloride, bromide, tetrachloroferrate, pentachlorostannate, hexafluorophosphate, butyltriphenylborate, or tetrakis(4-methoxyphenyl)borate.

### BRIEF SUMMARY OF THE DRAWINGS

FIG. 1 shows the proton NMR for the compound of Formula VII.

### DETAILED DESCRIPTION OF THE INVENTION

### I. General

The present disclosure describes methods of preparing 6-((1r,4r)-4-phenylcyclohexyl)-5-(3-(trifluoromethyl)benzyl)pyrimidine-2,4(1H,3H)-dione (Formula I), Example 6 of U.S. Pat. No. 8,685,973 via formation of a pyrylium intermediate from the compound of Formula II. The present disclosure also describes new intermediates.

### II. Definitions

“About” when referring to a value includes the stated value +/−10% of the stated value. For example, about 50% includes a range of from 45% to 55%, while about 10 equivalents includes a range of from 9 to 11 equivalents. Accordingly, when referring to a range, “about” refers to each of the stated values +/−10% of the stated value of each end of the range. For instance, a ratio of from about 1 to about 10 (w/w) includes a range of from 0.9 to 11.

“Forming a reaction mixture” refers to the process of bringing into contact at least two distinct species such that they mix together and can react. It should be appreciated, however, the resulting reaction product can be produced directly from a reaction between the added reagents or from an intermediate from one or more of the added reagents which can be produced in the reaction mixture.

“Solvent” refers to a substance, such as a liquid, capable of dissolving a solute. Solvents can be polar or non-polar, protic or aprotic. Polar solvents typically have a dielectric constant greater than about 5 or a dipole moment above about 1.0, and non-polar solvents have a dielectric constant below about 5 or a dipole moment below about 1.0. Protic solvents are characterized by having a proton available for removal, such as by having a hydroxy or carboxy group. Aprotic solvents lack such a group. Representative polar protic solvents include alcohols (methanol, ethanol, propanol, isopropanol, etc.), acids (formic acid, acetic acid, etc.) and water. Representative polar aprotic solvents include dichloromethane, chloroform, 1,4-dioxane, tetrahydrofuran, diethyl ether, acetone, ethyl acetate, N,N-dimethylformamide, dimethylacetamide, acetonitrile and dimethyl sulfoxide. Representative non-polar solvents include alkanes (pentanes, hexanes, etc.), cycloalkanes (cyclopentane, cyclohexane, etc.), benzene, and toluene. Other solvents are useful in the present invention.

“Acid” refers to a compound capable of donating a proton (a Bronsted-Lowry acid) or capable of accepting an electron pair (a Lewis acid). Representative acids include, but are not limited to, hydrochloric acid, sulfuric acid, formic acid, acetic acid, propanoic acid, butyric acid, hexanoic acid, octanoic acid, trifluoroacetic acid, tetrafluoroboric acid (HBF₄), etc.

“Strong acid” refers to an acid that easily dissociates, which is often represented by a pKₐ less than −1 in water. Representative strong acids include, but are not limited to, hydrochloric acid, hydrobromic acid, hydroiodic acid, nitric acid, sulfuric acid, and perchloric acid.

“Hydrate” refers to a compound that is complexed to at least one water molecule. The compounds of the present invention can be complexed with from 1 to 10 water molecules.

“Cooling” refers to applying cooling means to the reaction mixture to decrease the temperature of the reaction mixture by at least 1 degree Celsius. For example, cooling can include, but is not limited to, decreasing the temperature of the reaction mixture to or below room temperature.

“Heating” refers to applying heat to the reaction mixture to increase the temperature of the reaction mixture by at least 1 degree Celsius. For example, heating can include, but is not limited to, raising the temperature of the reaction mixture to room temperature, or to the reflux or boiling temperature of the reaction mixture, or to a temperature between room temperature and the reflux or boiling temperature of the reaction mixture.

“Room temperature” is the range of air temperatures generally considered to be suitable for human occupancy, or between about 15 degrees Celsius (59 degrees Fahrenheit) and 25 degrees Celsius (77 degrees Fahrenheit).

“Crystalline seed” refers to a seed crystal of the target crystalline form to be prepared.

### III. Pyrylium Method of Preparing Formula I

The present invention provides methods for the preparation of 6-((1r,4r)-4-phenylcyclohexyl)-5-(3-(trifluoromethyl)benzyl)pyrimidine-2,4(1H,3H)-dione, the compound of Formula I:

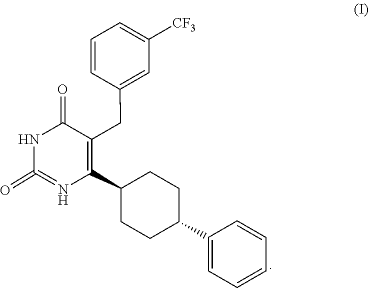

The compound of Formula I was originally disclosed as Example 6 in U.S. Pat. No. 8,685,973.

A. Preparation of Formula I from Formula VII

In some embodiments, the present invention provides a method of preparing a compound of Formula I:

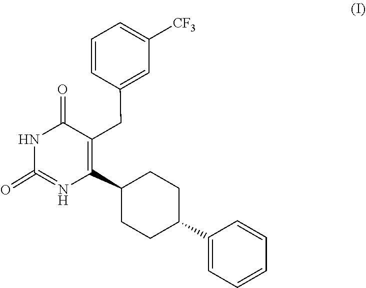

the method comprising: (a) forming a first reaction mixture comprising a first solvent, a strong acid, and a compound of Formula VII:

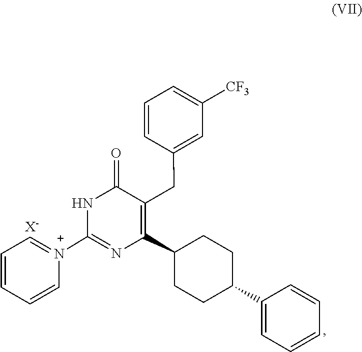

under conditions suitable to prepare the compound of Formula I, wherein counterion X⁻ is tetrafluoroborate, chloride, bromide, tetrachloroferrate, pentachlorostannate, hexafluorophosphate, butyltriphenylborate, or tetrakis(4-methoxyphenyl)borate.

Any suitable solvent can be used as the first solvent in the first reaction mixture of the present invention. Representative solvents include, but are not limited to, a polar aprotic solvent, a polar protic solvent, or a non-polar solvent. In some embodiments, the method of preparing the compound of Formula I includes the method wherein the first solvent comprises acetic acid, propionic acid, butyric acid, valeric acid, caproic acid, isobutyric acid, isovaleric acid, 4-methylvaleric acid, or 2-ethylcaproic acid. In some embodiments, the method of preparing the compound of Formula I includes the method wherein the first solvent comprises acetic acid, propionic acid, or butyric acid. In some embodiments, the method of preparing the compound of Formula I includes the method wherein the first solvent comprises acetic acid.

Any strong acid can be used as the strong acid in the first reaction mixture of the present invention. Representative strong acids include, but are not limited to, organic acids and inorganic acids. In some embodiments, the method of preparing the compound of Formula I includes the method wherein the strong acid comprises trifluoroacetic acid, trichloroacetic acid, ethane-1,2-disulfonic acid, naphthalene-2-sulfonic acid, naphthalene-1,5-disulfonic acid, hydrofluoric acid, hydrochloric acid, hydrobromic acid, hypochlorous acid, chloric acid, perchloric acid, sulfuric acid, nitric acid, phosphoric acid, methanesulfonic acid, ethanesulfonic acid, benzenesulfonic acid, p-toluenesulfonic acid, trifluoromethanesulfonic acid, camphorsulfonic acid, or combinations thereof. In some embodiments, the method of preparing the compound of Formula I includes the method wherein the strong acid comprises hydrochloric acid. In some embodiments, the method of preparing the compound of Formula I includes the method wherein the strong acid comprises concentrated hydrochloric acid. Concentrated hydrochloric acid can have a normality of about 12N and a concentration of about 12M. In some embodiments, the method of preparing the compound of Formula I includes the method wherein the strong acid comprises about 37% (w/w) hydrochloric acid.

The strong acid can be present in any suitable amount to the compound of Formula VII. For example, the strong acid can be present in an amount of from 1 to 10 molar equivalents to the compound of Formula VII, or from 1 to 8, from 2 to 7, from 4 to 6, from 4.5 to 5.5, from 4.6 to 5.4, from 4.7 to 5.3, from 4.8 to 5.2, or from 4.9 to 5.1 molar equivalents to the compound of Formula VII. Representative amounts of the strong acid include, but are not limited to, about 1.0 molar equivalent to the compound of Formula VII, or about 2.0, 3.0, 3.5, 4.0, 4.5, 4.6, 4.7, 4.8, 4.9, 5.0, 5.1, 5.2, 5.3, 5.4, 5.5, 6.0, 6.5, 7.0, 8.0, 9.0, or about 10 molar equivalents to the compound of Formula VII. In some embodiments, the method of preparing the compound of Formula I includes the method wherein the strong acid is present in an amount from 4 to 6 molar equivalents to the compound of Formula VII. In some embodiments, the method of preparing the compound of Formula I includes the method wherein the strong acid is present in an amount of about 5 molar equivalents to the compound of Formula II.

Any suitable counterion can be used as the counterion X⁻ of the compound of Formula VII in the first reaction mixture of the present invention. Representative counterions X⁻ include tetrafluoroborate, chloride, bromide, tetrachloroferrate, pentachlorostannate, hexafluorophosphate, butyltriphenylborate, or tetrakis(4-methoxyphenyl)borate. In some embodiments, the method of preparing the compound of Formula I includes the method wherein counterion X⁻ is BF₄⁻.

In some embodiments, the method of preparing the compound of Formula I includes the method wherein the compound of Formula VII has the structure:

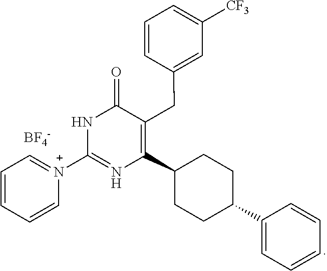

In some embodiments, the method of preparing the compound of Formula I includes the method comprising: (a) forming the first reaction mixture comprising the compound of Formula VII, acetic acid, and concentrated hydrochloric acid, under conditions suitable to prepare the compound of Formula I.

B. Preparation of Formula VII from Formula II

The compound of Formula VII, a salt comprising 1-(6-oxo-4-((1r,4r)-4-phenylcyclohexyl)-5-(3-(trifluoromethyl)benzyl)-1,6-dihydropyrimidin-2-yl)pyridin-1-ium:

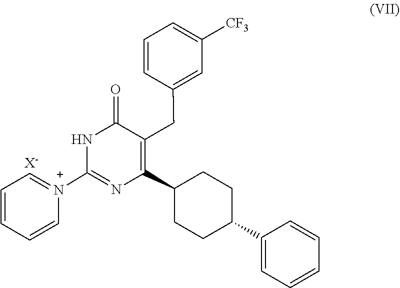

can be prepared by a variety of methods.

In some embodiments, the present invention provides a method of preparing a compound of Formula VII:

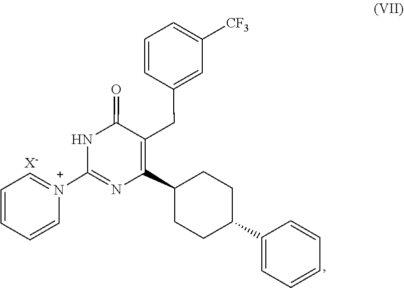

comprising
   (b) forming a second reaction mixture comprising a pyrylium salt:

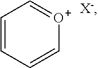

a second solvent and a compound of Formula II, or a hydrate thereof:

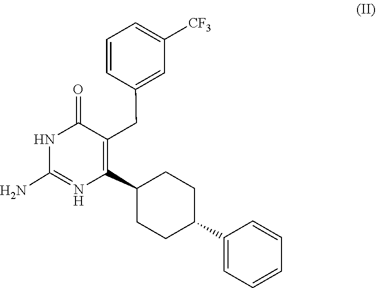 [SMILES: Nc1nc([C@H]2CC[C@H](c3ccccc3)CC2)c(Cc2cccc(C(F)(F)F)c2)c(=O)[nH]1]

under conditions suitable to prepare the compound of Formula VII,
wherein counterion X⁻ is BF₄⁻.

In some embodiments, the method of preparing the compound of Formula VII includes the method wherein the pyrylium salt is pyrylium tetrafluoroborate having the structure:

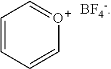

The pyrylium salt can be present in any suitable amount to the compound of Formula II. For example, the pyrylium salt can be present in an amount of from 0.1 to 10 molar equivalents to the compound of Formula II, or from 0.2 to 5, from 0.5 to 3, from 1 to 3, from 1 to 1.5, or from 1 to 1.2 molar equivalents to the compound of Formula II. Representative amounts of the pyrylium salt include, but are not limited to, about 0.5 molar equivalents to the compound of Formula II, or about 0.6, 0.7, 0.8. 0.9, 1.0, 1.1, 1.2, 1.3, 1.4, 1.5, 2.0, 2.5, 3.0, 4.0, 5.0, 6.0, 7.0, 8.0, 9.0, or 10 molar equivalents to the compound of Formula II. In some embodiments, the method of preparing the compound of Formula VII includes the method wherein the pyrylium salt is present in an amount of from 1 to 3 molar equivalents to the compound of Formula II. In some embodiments, the method of preparing the compound of Formula VII includes the method wherein the pyrylium salt is present in an amount of about 1.1 molar equivalents to the compound of Formula II.

Any suitable solvent can be used as the second solvent in the second reaction mixture of the present invention. Representative second solvents include, but are not limited to, polar protic solvents, polar aprotic solvents, and non-polar solvents. In some embodiments, the method of preparing the compound of Formula VII includes the method wherein the second solvent comprises methanol, ethanol, n-propanol, isopropanol, n-butanol, t-butanol, 1-pentanol, 2-pentanol, 3-pentanol, 2-methyl-1-propanol, 1-methoxy-2-propanol, 4-methyl-2-pentanol, 1-hexanol, 2-hexanol, 1-octanol, or 2-octanol, or combinations thereof. In some embodiments, the method of preparing the compound of Formula VII includes the method wherein the second solvent comprises n-propanol or n-butanol. In some embodiments, the method of preparing the compound of Formula VII includes the method wherein the second solvent comprises n-butanol.

The compound of Formula II can be the free base form or the hydrate form. In some embodiments, the compound of Formula II is the free base. In some embodiments, the compound of Formula II is the hydrate. The compound of Formula II can be any suitable hydrate. For example, the compound of Formula II can be a monohydrate, dihydrate, trihydrate, or tetrahydrate. In some embodiments, the compound of Formula II is a monohydrate.

In some embodiments, the method of preparing the compound of Formula VII includes the method wherein the compound of Formula II is the monohydrate form:

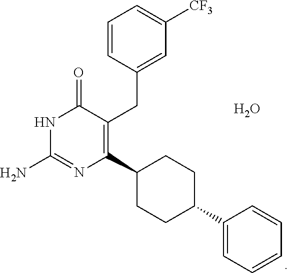 [SMILES: Nc1nc([C@H]2CC[C@H](c3ccccc3)CC2)c(Cc2cccc(C(F)(F)F)c2)c(=O)[nH]1 ; O]

In some embodiments, the method of preparing the compound of Formula VII includes the method comprising (b) forming the second reaction mixture comprising pyrylium tetrafluoroborate in an amount of about 1.1 molar equivalents to the compound of Formula II, n-butanol, and the compound of Formula II, under conditions suitable to prepare the compound of Formula VII.

In some embodiments, the present invention provides a method of preparing a compound of Formula I, wherein the compound of Formula VII is prepared by the methods of the present invention.

In some embodiments, the method of preparing the compound of Formula I includes the method comprising: (b) forming the second reaction mixture comprising pyrylium tetrafluoroborate in an amount of about 1.1 molar equivalents to the compound of Formula II, n-butanol, and the compound of Formula II, under conditions suitable to prepare the compound of Formula VII; and (a) forming the first reaction mixture comprising the compound of Formula VII, acetic acid, and concentrated hydrochloric acid, under conditions suitable to prepare the compound of Formula I.

### C. Pyrylium Intermediate Compounds

The present invention provides compounds of Formula VII. In some embodiments, the present invention provides a compound of Formula VII:

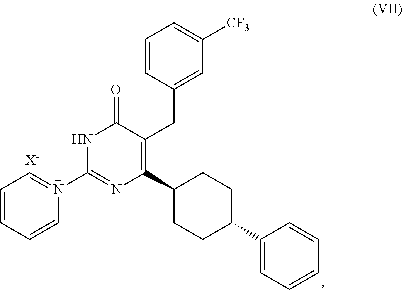

wherein counterion X⁻ is tetrafluoroborate, chloride, bromide, tetrachloroferrate, pentachlorostannate, hexafluorophosphate, butyltriphenylborate, or tetrakis(4-methoxyphenyl)borate.

In some embodiments, the compound of Formula VII has the structure:

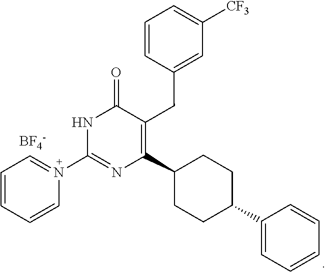

The compound of Formula VII is named 1-(6-oxo-4-((1r,4r)-4-phenylcyclohexyl)-5-(3-(trifluoromethyl)benzyl)-1,6-dihydropyrimidin-2-yl)pyridin-1-ium tetrafluoroborate using IUPAC nomenclature.

### IV. Examples

The following acronyms and abbreviations are used in the methods below:

| | |
|---|---|
| AcOH | Acetic acid |
| aq | Aqueous |
| atm | Atmospheric pressure |
| ° C. | Degree Celsius |
| DBU | 1,8-Diazabicyclo[5.4.0]undec-7-ene |
| DCM | Dichloromethane |
| DMF | N,N-Dimethylformamide |
| DMAP | N,N-Dimethylaminopyridine/4- |
| | (Dimethylamino)pyridine |
| DSC | Differential Scanning Calorimetry |
| EDC | 1-Ethyl-3-(3-dimethylaminopropyl)carbodiimide |
| eq | Equivalent |
| GF | Glass microfiber filter paper |
| g | Gram |
| HCl | Hydrochloric acid |
| hdpe | High density polyethylene |
| HPLC | High-performance Liquid Chromatography |
| KF | Karl Fischer titration |
| L | Liter |
| LCAP | HPLC Area Percent |
| LCWP | HPLC Weight Percent |
| M | Molar |
| MeCN | Acrylonitrile |
| MeOH | Methanol |
| mins | Minutes |
| mL | Milliliter |
| nBuOH | butan-1-ol |
| NMP | N-Methyl-2-pyrrolidone |
| Pd-C | Palladium on carbon |
| ¹H-NMR | Proton Nuclear Magnetic Resonance |
| ref std | Reference Standard |
| RT | Room Temperature |
| SM | Starting Material |
| T | Temperature |
| THF | Tetrahydrofuran |
| TMS | Tetramethylsilane |
| TG-DTA | Thermogravimetric Differential Thermal Analysis |
| vol | Volume |
| wrt | With respect to |
| Wt | Weight |
| XRPD | X-ray Powder Diffraction |

X-ray Powder Diffraction (XRPD). XRPD analyses were performed using a Panalytical Xpert Pro diffractometer equipped with a Cu X-ray tube and a Pixcel detector system. The samples were analyzed at ambient temperature in transmission mode and held between PVC films. The default XRPD program was used (range 3-40° 2θ, step size 0.013°, counting time 99 sec, ˜22 min run time/counting time 49 sec for the Compound of Formula II and ˜11 min run time/counting time 22 sec for the both Forms of the Compound of Formula VII Samples were spun at 60 rpm during data collection. XRPD patterns were sorted, manipulated using HighScore Plus 2.2c software.

Differential Scanning Calorimetry (DSC). DSC analyses were carried out on a Perkin Elmer Jade Differential Scanning Calorimeter. Accurately weighed samples were placed in gold pans and lid secured. Each sample was heated under nitrogen at a rate of 5° C./minute to a maximum of 200 or 300° C.

Thermogravimetric Differential Thermal Analysis (TG-DTA). Thermogravimetric (TG) analyses were carried out on a Mettler Toledo TGA/DSC 1 STAR^{e} simultaneous thermal analysis instrument. Samples were placed in an aluminium sample pan, inserted into the TG furnace and accurately weighed. Under a stream of nitrogen at a rate of 10° C./minute, the heat flow signal was stabilized for one minute at 30° C., prior to heating to 300° C.

Proton Nuclear Magnetic Resonance Spectroscopy (¹H-NMR). Proton NMR analyses were performed on a 500 MHz Bruker AVANCE NEO instrument fitted with a Prodigy BBO CryoProbe. Samples were dissolved in the appropriate deuterated NMR solvent containing tetramethylsilane (TMS) as an internal standard, possessing an isotopic purity of ≥99.5 atom % D, then the solution of the sample was analyzed using 5 mm Virgin NMR tubes.

“Conditions suitable” for conducting the methods of the present invention include the time and temperature for conducting the methods, as defined below.

The reaction steps of the present invention can be performed for any suitable reaction time. For example, the reaction time can be for minutes, hours, or days. In some embodiments, the reaction time can be for several hours, such as at least eight hours. In some embodiments, the reaction time can be for several hours, such as at least overnight. In some embodiments, the reaction time can be for several days. In some embodiments, the reaction time can be for at least two hours. In some embodiments, the reaction time can be for at least eight hours. In some embodiments, the reaction time can be for at least several days. In some embodiments, the reaction time can be for about two hours, or for about 4 hours, or for about 6 hours, or for about 8 hours, or for about 10 hours, or for about 12 hours, or for about 14 hours, or for about 16 hours, or for about 18 hours, or for about 20 hours, or for about 22 hours, or for about 24 hours. In some embodiments, the reaction time can be for about 1 day, or for about two days, or for about three days, or for about four days, or for about five days, or for about six days, or for about a week, or for about more than a week.

The reaction steps of the present invention can be performed at any suitable reaction temperature. Representative temperatures include, but are not limited to, below room temperature, at room temperature, or above room temperature. Other temperatures useful in the methods of the present invention include from about −40° C. to about 65° C., or from about room temperature to about 40° C., or from about 40° C. to about 65° C., or from about 40° C. to about 60° C. In some embodiments, the reaction mixture can be at a temperature of about room temperature, or at a temperature of about 15° C., or at about 20° C., or at about 25° C. or at about 30° C., or at about 35° C., or at about 40° C., or at about 45° C., or at about 50° C., or at about 55° C., or at about 60° C., or at about 65° C.

### Example 1. Preparation of 6-((1r,4r)-4-phenylcyclohexyl)-2-(1M-pyrylium-1-yl)-5-(3-(trifluoromethyl)benzyl)pyrimidin-4(3H)-one tetrafluoroboronate (Formula VII)

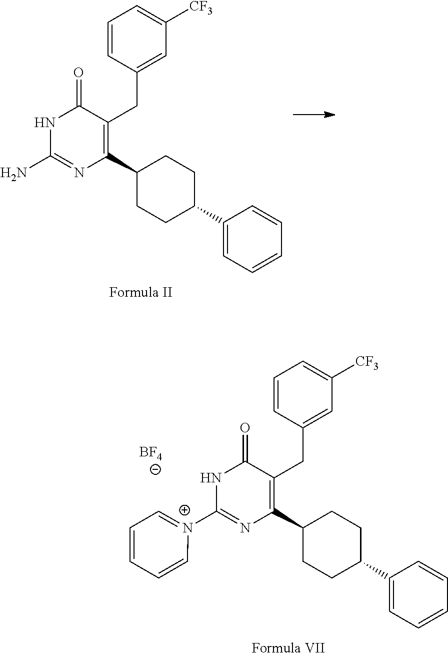 [SMILES: Nc1nc([C@H]2CC[C@H](c3ccccc3)CC2)c(Cc2cccc(C(F)(F)F)c2)c(=O)[nH]1 ; O=c1[nH]c(-[n+]2ccccc2)nc([C@H]2CC[C@H](c3ccccc3)CC2)c1Cc1cccc(C(F)(F)F)c1 ; "BF4"]

Formula II was prepared according to the methods described in WO2022/140293.

Formula II (33 g, 94 wt %, 72.6 mmol, 1 eq.) was slurried in nBuOH (42 vol., 1386 ml). The stream was concentrated to 35 vol. (1155 ml) under vacuum. The stream was transferred to an inerted jacketed vessel equipped with an overhead stirrer and treated with Pyrylium·BF₄ (11 g, 97.7 wt %, 65.3 mmol, 0.9 eq.). The contents were heated to 100° C. and aged for 2 hours. The reaction was analysed by HPLC, indicating 16.0 LCAP of Formula II still present.

The stream was distilled to 30 vol. under vacuum, nBuOH (5 vol., 165 ml) was added followed by Pyrylium·BF₄ (1.21 g, 7.25 mmol, 0.1 eq.). After aging at 100° C. for 1 hour, HPLC indicated 7.7 LCAP SM remaining. The stream was distilled to 30 vol. and nBuOH (5 vol., 165 ml) was added, followed by Pyrylium·BF₄ (0.61 g, 0.05 eq., 3.62 mmol). After a 1-hour age at 100° C., HPLC indicated that 5.6 LCAP of Formula II remained.

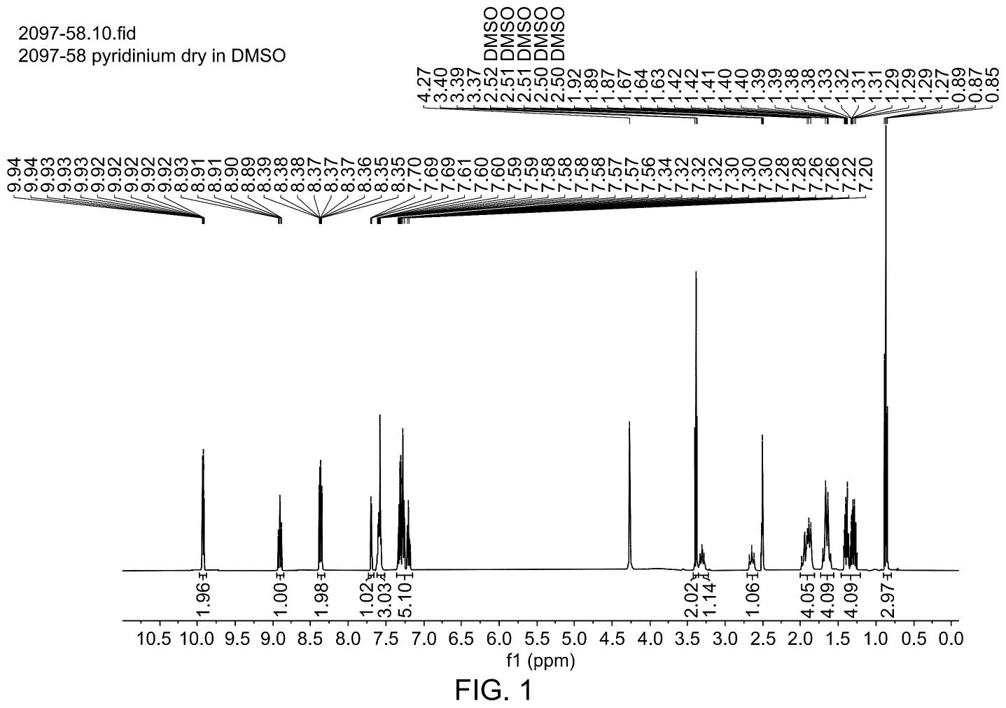

The stream was distilled to a final volume of 5 vol. (165 ml) and the resultant slurry was aged overnight. The liquor concentration of Formula VII was determined at 8.2 mg/ml. The product was isolated via filtration and the cake was washed with nBuOH (2×2 vol., 66 ml). The product was transferred to a vacuum oven and dried overnight at 50° C. A total of 39.1 g of Formula VII was isolated as a beige crystalline solid. m/z [M+H]: 490.30. ¹⁹F NMR (DMSO-d6): −61.1, −148.3. The ¹H NMR (DMSO-d6) is provided in FIG. 1.

### Example 2. Preparation of 6-((1r,4r)-4-phenylcyclohexyl)-5-(3-(trifluoromethyl)benzyl)pyrimidine-2,4(1H,3H)-dione (Formula I)

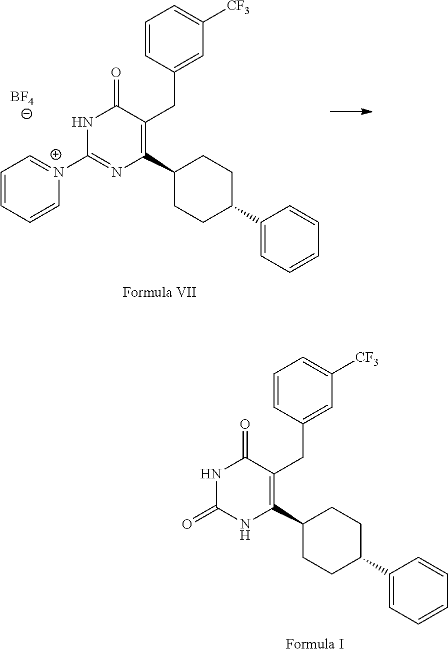 [SMILES: O=c1[nH]c(-[n+]2ccccc2)nc([C@H]2CC[C@H](c3ccccc3)CC2)c1Cc1cccc(C(F)(F)F)c1 ; "BF4" ; O=c1[nH]c(=O)c(Cc2cccc(C(F)(F)F)c2)c([C@H]2CC[C@H](c3ccccc3)CC2)[nH]1]

Formula VII (72.2 g, 93.6 wt %, 117.1 mmol, 1.0 eq.) was charged to an inerted 1 L round-bottom flask fitted with an overhead stirrer. AcOH (8 vol., 578 ml) was added, and the contents were stirred. To the slurry was added cone HCl (˜37%, 48.8 ml, 58.1 g, 585.5 mmol, 5 eq.). The contents were warmed to 70° C. and aged. The reaction was monitored by HPLC for extent of conversion.

After 3 hours the reaction was deemed complete and the contents were cooled to 20° C., over ˜1 hour, during which time a slurry formed. To the slurry was added water (1 vol., 72 ml), drop-wise over ˜1 hour. The slurry was aged for 30 mins and the liquor concentration of Formula I was determined as 2.8 mg/ml. The slurry was aged for a further 1 hour and the liquor loss dropped to 2.5 mg/ml. The product was isolated via filtration and the filter cake was washed sequentially with AcOH:water (8:1, 2×2 vol., 144 ml) and water (2×2 vol., 144 ml). The product was transferred to a vacuum oven and dried overnight at 50° C. CORT118335 (miricorilant) (52.8 g) was isolated as an off-white crystalline solid. 1H-NMR was consistent with Formula I standard, and product appeared to be an AcOH solvate.

Formula I (50 g, 89.7 wt %, 104.7 mmol, 1 eq.) was slurried in DCM (11 vol., 550 ml) in an inerted 1 L round-bottom flask equipped with an overhead stirrer. The slurry was warmed to 30° C., forming a solution. The solution was filtered through GF/A grade filter paper (coloured imps removed) and a line wash of DCM (1 vol., 50 ml) was conducted. The DCM streams were combined, and MeOH (3 vol., 150 ml) added. The solution was distilled to a final volume of 5 vol. (250 ml) at atmospheric pressure (T=˜50° C.). To the stream was added MeOH (500 ml, 10 vol.) and the solution was seeded with authentic Formula I (0.1 wt %, 50 mg, 0.105 mmol), generating a mobile white slurry which thickened over time. The slurry was distilled to a final volume of 5 vol. (250 ml) under atmospheric pressure (T˜60° C.), and the stream was treated with MeOH (500 ml, 10 vol.). The stream was distilled to a final volume of 5 vol. (250 ml), under atmospheric pressure (T˜65° C.). The slurry was treated with MeOH (250 ml, 5 vol.) and the batch was allowed to cool to 20-25° C. over a period of two hours. A sample of the slurry was removed and analysed via XRPD confirming the correct polymorphic form had been generated. The liquor concentration of miricorilant was determined as 4.1 mg/ml and the product was isolated by filtration and the cake was washed with MeOH (2×1 vol., 50 ml). The product was transferred to a vacuum oven and dried overnight at 50° C. Pure Formula I was obtained as a white crystalline solid (42.4 g).

The characterization data of the title product matched those of Example 6 of U.S. Pat. No. 8,685,973, and Example 2 of U.S. Pat. No. 11,548,856.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of preparing a compound of Formula I: 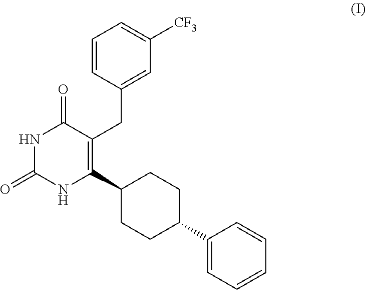 [SMILES: O=c1[nH]c(=O)c(Cc2cccc(C(F)(F)F)c2)c([C@H]2CC[C@H](c3ccccc3)CC2)[nH]1]
the method comprising:
(a) forming a first reaction mixture comprising a first solvent, a strong acid, and a compound of Formula VII: 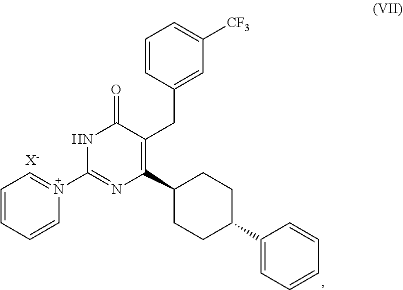
under conditions suitable to prepare the compound of Formula I,
wherein counterion X⁻ is tetrafluoroborate, chloride, bromide, tetrachloroferrate, pentachlorostannate, hexafluorophosphate, butyltriphenylborate, or tetrakis(4-methoxyphenyl)borate.

2. The method of claim 1, wherein the first solvent comprises acetic acid, propionic acid, butyric acid, valeric acid, caproic acid, isobutyric acid, isovaleric acid, 4-methylvaleric acid, or 2-ethylcaproic acid.

3. The method of claim 1, wherein the strong acid comprises trifluoroacetic acid, trichloroacetic acid, ethane-1,2-disulfonic acid, naphthalene-2-sulfonic acid, naphthalene-1,5-disulfonic acid, hydrofluoric acid, hydrochloric acid, hydrobromic acid, hypochlorous acid, chloric acid, perchloric acid, sulfuric acid, nitric acid, phosphoric acid, methanesulfonic acid, ethanesulfonic acid, benzenesulfonic acid, p-toluenesulfonic acid, trifluoromethanesulfonic acid, camphorsulfonic acid, or combinations thereof.

4. The method of claim 1, wherein the strong acid comprises hydrochloric acid.

5. The method of claim 1, wherein counterion X⁻ is BF₄⁻.

6. The method of claim 1, wherein the compound of Formula VII has the structure:

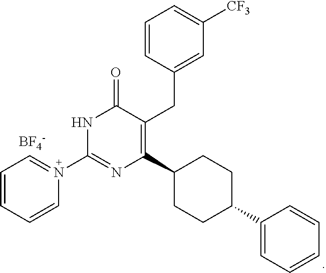

7. The method of claim 1, comprising:
(a) forming the first reaction mixture comprising the compound of Formula VII, acetic acid, and concentrated hydrochloric acid, under conditions suitable to prepare the compound of Formula I.

8. A method of preparing a compound of Formula VII: 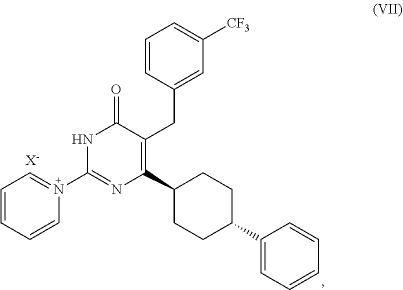
comprising
(b) forming a second reaction mixture comprising a pyrylium salt: 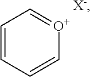
a second solvent and a compound of Formula II, or a hydrate thereof: 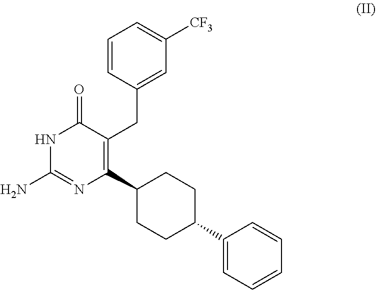 [SMILES: Nc1nc([C@H]2CC[C@H](c3ccccc3)CC2)c(Cc2cccc(C(F)(F)F)c2)c(=O)[nH]1]
under conditions suitable to prepare the compound of Formula VII, wherein counterion X⁻ is BF₄⁻.

9. The method of claim 8, wherein the pyrylium salt is pyrylium tetrafluoroborate having the structure:

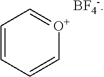

10. The method of claim 8, wherein the pyrylium salt is present in an amount of from 1 to 3 molar equivalents to the compound of Formula II.

11. The method of claim 8, wherein the pyrylium salt is present in an amount of about 1.1 molar equivalents to the compound of Formula II.

12. The method of claim 8, wherein the second solvent comprises methanol, ethanol, n-propanol, isopropanol, n-butanol, t-butanol, 1-pentanol, 2-pentanol, 3-pentanol, 2-methyl-1-propanol, 1-methoxy-2-propanol, 4-methyl-2-pentanol, 1-hexanol, 2-hexanol, 1-octanol, or 2-octanol, or combinations thereof.

13. The method of claim 8, wherein the second solvent comprises n-propanol or n-butanol.

14. The method of claim 8, wherein the compound of Formula II is the monohydrate form:

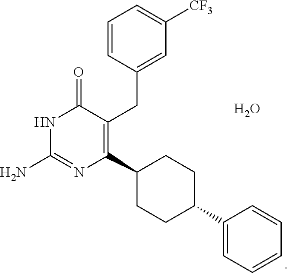 [SMILES: Nc1nc([C@H]2CC[C@H](c3ccccc3)CC2)c(Cc2cccc(C(F)(F)F)c2)c(=O)[nH]1 ; O]

15. The method of claim 8, comprising
(b) forming the second reaction mixture comprising pyrylium tetrafluoroborate in an amount of about 1.1 molar equivalents to the compound of Formula II, n-butanol, and the compound of Formula II, under conditions suitable to prepare the compound of Formula VII.

16. The method of claim 1, wherein the compound of Formula VII is prepared by
(b) forming a second reaction mixture comprising a pyrylium salt: 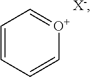
a second solvent and a compound of Formula II, or a hydrate thereof: 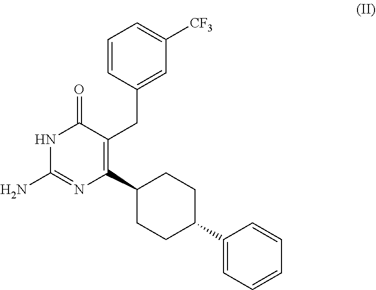 [SMILES: Nc1nc([C@H]2CC[C@H](c3ccccc3)CC2)c(Cc2cccc(C(F)(F)F)c2)c(=O)[nH]1]
under conditions suitable to prepare the compound of Formula VII, wherein counterion X⁻ is BF₄⁻.

17. The method of claim 16, comprising:
(b) forming the second reaction mixture comprising pyrylium tetrafluoroborate in an amount of about 1.1 molar equivalents to the compound of Formula II, n-butanol, and the compound of Formula II, under conditions suitable to prepare the compound of Formula VII; and
(a) forming the first reaction mixture comprising the compound of Formula VII, acetic acid, and concentrated hydrochloric acid, under conditions suitable to prepare the compound of Formula I.

18. A compound of Formula VII: 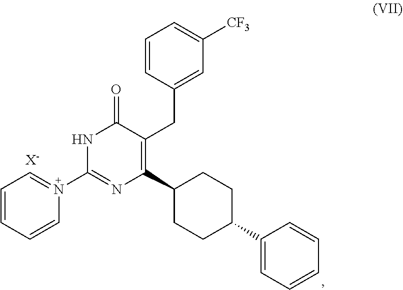
wherein counterion X⁻ is tetrafluoroborate, chloride, bromide, tetrachloroferrate, pentachlorostannate, hexafluorophosphate, butyltriphenylborate, or tetrakis(4-methoxyphenyl)borate.

19. The compound of claim 18, wherein the compound of Formula VII has the structure: [chemical structure]
